(19) United States
(12) Patent Application Publication
(10) Pub. No.: US 2007/0270657 A1
(43) Pub. Date: Nov. 22, 2007

(54) External Eye Speculum

(76) Inventors: John Stephenson, Morrison, CO (US); Barry Schafer, El Dorado Hills, CA (US)

Correspondence Address: BROWNSTEIN HYATT FARBER SCHRECK, PC, 410 SEVENTEENTH STREET, SUITE 2200, DENVER, CO, 80202, US

(21) Appl. No.: 11/308,879

(22) Filed: May 19, 2006

(51) Int. Cl. A61B 1/32

(52) U.S. Cl. 600/236

(57) ABSTRACT

An external eye speculum for use in connection with, for example, examination, testing, treatment, surgery, and contact lens fitting and placement comprising a first arm and a second arm, the first arm and the second arm each have an open end and a closed end, the closed end of the first arm and the closed end of the second arm are joined to one another at a point. The first arm is adapted to receive an outer portion of an upper eye lid and the second arm is adapted to receive an outer portion of a lower eye lid. An adhesive is affixed to at least a portion of the first arm and the second arm and reversibly adheres to the upper eye lid and the lower eye lid to further to hold the speculum in place and provide additional eye lid and lash support. Raised members found on the first arm and second arm serve as holds so that pressure can be applied to the first arm and second arm causing the inventive device to move to a closed position. Optionally, a handle may be employed which preferably extends in a generally outwardly direction from the point.

### TECHNICAL FIELD

The present invention relates to eye speculums for use in connection with, for example, examination, testing, treatment, and contact lens fitting and placement, and, more particularly, to an external eye speculum that enables an examiner to perform eye examinations, testing, treatment, and contact lens fitting and placement without having to place the speculum within any part of the eye or surrounding eye structures.

### BACKGROUND OF THE INVENTION AND PRIOR ART

When performing eye examinations, eye lids and lashes get in the way of an unobstructed view of the eye. Unobstructed views are critical to accurate eye examination and testing, eye injections, contact lens insertion and removal, and extraction of a foreign body. Failure to have an unobstructed view of the eye can lead to incomplete examination, misdiagnosis, and increased liability for the examiner.

In an effort to create an unobstructed view examiners have tried various methods over the years. For example, they can choose to hold the eyelids open with his or her fingers, or have an assistant do so. Holding the eyelids open and also performing the exam or test (hereinafter the use of the exam or test, or variants of each word, is meant to include both exam or test, or variant, unless noted otherwise) is cumbersome and can lead to difficulties in the examination and increases the risk of error. Having an assistant hold an eyelid open is better but is also cumbersome and the assistant may get in the way of the examiner. It is also not uncommon for an examiner to tape the eyelids of the patient out of the way.

Another option is the use of an eye speculum. Speculums are designed to hold the eyelids open such that the eye can be clearly visualized and an examination can be accomplished efficiently. Prior art eye speculums are termed internal eye speculums. Internal eye speculums are generally used in surgery to retract the eyelids and lashes. Numbing drops are usually required when setting an internal eye speculum as they are very uncomfortable for the patient. Moreover, corneal epithelial abrasion is a real risk in the use of an internal eye speculum. In addition, sterilization of internal eye speculums is required.

The frustration with having lids and lashes in the way has always been a problem and is felt industry wide. Physicians and equipment manufacturers alike are searching for a viable solution. Accordingly, there is need for an external eye speculum that efficiently holds the eyelids and lashes away from the eye during an examination. The device should be easy to use and not require sterilization, the use of drugs, or increase the risk of eye damage. The present inventive external eye speculum meets these needs and others.

### OBJECTS AND ADVANTAGES OF THE PRESENT INVENTION

It is an object of the present invention to provide an external eye speculum that is set on the exterior of the eye and efficiently holds the eyelids and lashes away from the eye.

It is a further object of the present invention to provide an external eye speculum that is easy to use.

It is yet a further object of the present invention to provide an external eye speculum that will have an adhesive that will help in raising and holding the lids and lashes out of the way during the eye test.

The advantages offered by the present invention include but are not limited to solving a real problem of obstruction of the eye during an examination through the use of the inventive external eye speculum which will decrease examination time, increase examination and test accuracy, decrease test false positives and negatives, decrease retesting, increase diagnosis accuracy, and potentially decrease liability.

### SUMMARY OF THE INVENTION

According to the invention, an external eye speculum device is presented. The inventive device comprises a first arm and a second arm. The first arm and the second arm each have an open end and a closed end, the closed end of the first arm and the closed end of the second arm are joined to one another at a point. The first arm is adapted to receive an outer portion of an upper eye lid and the second arm is adapted to receive an outer portion of a lower eye lid.

An adhesive is affixed to at least a portion of the first arm and the second arm. The adhesive reversibly adheres to the upper eye lid and the lower eye lid to further to hold the speculum in place and provide additional eye lid and lash support.

A first raised member is found on the first arm and a second raised member is found on the second arm. The first and second raised members serve as holds so that pressure can be applied to the first arm and second arm causing the inventive device to move to a closed position.

Optionally, a handle may be employed which preferably extends in a generally outwardly direction from the point.

In use, a paper backing which covers the adhesive found on the inventive external eye speculum is removed. The first and second raised members are then pinched towards one another thereby causing the inventive device to move to a closed position. The device is then placed over the target eye to a point that it contacts the eyelid, and the adhesive will reversibly adhere to the eye lid and then lash. Further deployment of the inventive device through the continued release of pressure will cause the eye lid and lash to move from the eye resulting in an unobstructed view of the target eye.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

As such, those skilled in the art will appreciate that the conception, upon which this disclosure is based, may readily be utilized as a basis for designing of other structures, methods, and systems for carrying out the several purposes of the present invention. It is important, therefore, that the claims be regarded as including such equivalent constructions in so far as they do not depart from the spirit and scope of the present invention.

Further, the purpose of the abstract is to enable the US patent and trademark office and the public generally, and especially the scientists, engineers and practitioners in the art who are not familiar with the patent or legal terms or phraseology, to determine quickly from what cursory inspection the nature and essence of the technical disclosure of the application. The abstract is neither intended to define the invention of the application, which is measured by the claims, nor is it intended to be limiting as to the scope of the present invention in any way.

These together with other objects of the present invention, along with the various features of novelty which characterize the present invention, are pointed out with particularity in the claims annexed to and forming a part of this disclosure. For a better understanding of the present invention, its operating advantages and the specific objects attained by its uses, reference should be had to the accompanying drawings and descriptive matter in which there is illustrated a preferred embodiment of the present invention.

Before explaining the preferred embodiment of the present invention in detail, it is to be understood that the present invention is not limited in its application to the details of construction and to the arrangements of the components set forth in the following description or illustrated in the drawings. The present invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1 depicts a top view of the inventive device.

FIG. 2 depicts a bottom view of the inventive device.

FIG. 3 depicts a back view of the inventive device.

### DETAILED DESCRIPTION OF THE PREFERRED EMBODIMENT

Before explaining the preferred embodiment of the present invention in detail, it is to be understood that the present invention is not limited in its application to the details of construction and to the arrangements of the components set forth in the following description or illustrated in the drawings. The present invention is capable of other embodiments and of being practiced and carried out in various ways. Also, it is to be understood that the phraseology and terminology employed herein are for the purpose of description and should not be regarded as limiting.

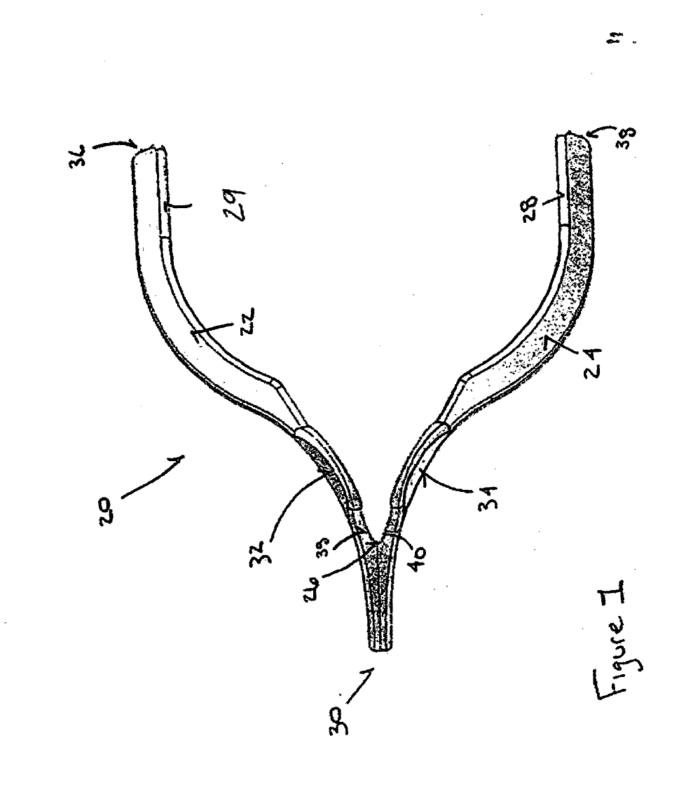

Turning to FIG. 1, it is seen that inventive external eye speculum (“device”) 20 comprises a first arm 22 and a second arm 24, first arm 22 and second arm 24 each have an open end 38, 36 and a closed end 33, 40. Closed end 33 of first arm 22 and closed end 40 of second arm 24 are joined to one another at a point 26.

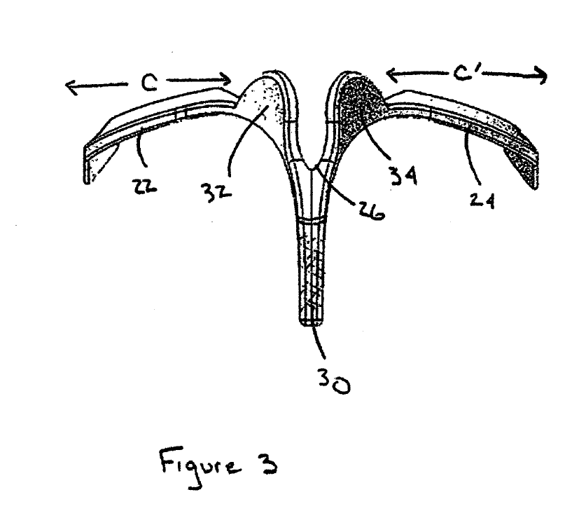

As seen in FIG. 3, first arm 22 and second arm 24 have a length, C and C′. Lengths C and C′ are of a length such that they fit along the shape of the eye.

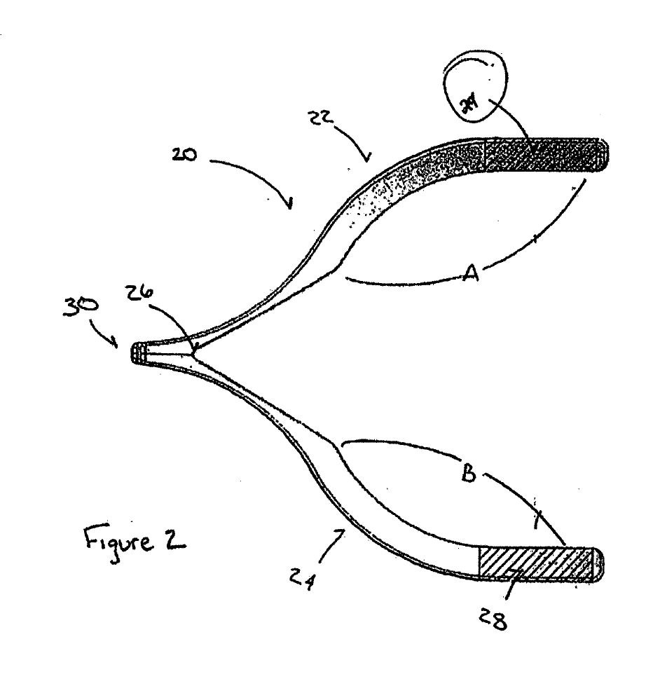

As seen in FIG. 2, an adhesive 29, 28 is affixed to at least a portion of the length C of the first arm 22 and C′ of the second arm 24. The adhesive reversibly adheres to the upper eye lid and the lower eye lid to further to hold device 20 in place and provide additional eye lid and lash support.

As seen in FIG. 2 and FIG. 3, a first raised member 32 is found on the first arm 22 and a second raised member 34 is found on the second arm 24. The first 32 and second 34 raised members serve as holds so that pressure can be applied to the first arm 22 and second arm 24.

Optionally, a handle 30 may be employed which preferably extends in a generally outwardly direction from the point 26. Handle 30 may be useful in further manipulation of device 20 to set it into proper place.

Turning to FIG. 2, it is seen that in the preferred embodiment first arm 22 and second arm 24 are generally curvilinear in shape along axis A and B, respectively. The curvilinear shape of first arm 22 and second arm 24 is to fit around the generally circular shape of an optic orbit.

In use, a protective covering found on inventive external eye speculum adhesive 29, 28 is removed. The first 32 and second 34 raised members are then pinched towards one another thereby causing the inventive device to move to a closed position. Device 20 is then placed over the target eye and the pressure pinching the first raised member 32 and second raised member 34 is slowly released thereby deploying the inventive device.

Once the first 22 and second 24 arms have deployed to a point that they contact the eyelid, the adhesive 26, 28 will reversibly adhere to the eye lid and then lash. Further deployment of the inventive device through the continued release of pressure will cause the eye lid and lash to open, resulting in an unobstructed view of the target eye.

It is to be understood, however, that even though numerous characteristics and advantages of the preferred and alternative embodiments have been set forth in the foregoing description, together with details of the structure and function of the embodiments, the disclosure is illustrative only.

For non-limiting example, the inventors contemplate that the device 20 will be of the preferred design above. However, any general shape of the first arm and second arm can be used. For example, it is conceivable that instead of curvilinear, first and second arms could be straight causing the device to have a “V” shape instead of a generally “U” shape.

Moreover, it is contemplated that the device 20 will be a single patient use device made of a plastic. However, it is conceivable that the device could be reusable and made of materials other than plastic, such as metal.

Such variations should be considered to be within the scope and spirit of the invention which is meant to cover an eye speculum which is useful for the examination, testing, and treatment of an eye by enabling an examiner to place the speculum external to the eye and lashes yet open the eye lid. Changes may be made in detail, especially in matters of shape, size and arrangement of parts within the principles of the invention to the full extent indicated by the broad general meaning of the terms in which the appended claims are expressed.

What is claimed is:

1. An eye speculum comprising a first arm and a second arm, the first arm and the second arm each having an open end and a closed end, the first arm and the second arm further being of a predetermined length, the closed end of the first arm and the closed end of the second arm being joined to one another at a point, wherein the first arm is adapted to receive an outer portion of an upper eye lid and the second arm is adapted to receive an outer portion of a lower eye lid.

2. The external eye speculum of claim 1 further comprising adhesive affixed to at least a portion of the length of at least one of the first arm and the second arm, the adhesive for reversibly adhering to the upper eye lid and the lower eye lid.

3. The external eye speculum of claim 2 wherein the adhesive is tape.

4. The external eye speculum of claim 1 further comprising a handle extending in a generally outwardly direction from the point.

5. The external eye speculum of claim 1 further comprising a first raised member extending generally upward direction from the first arm and a second raised member extending generally upward direction from the second arm.

6. The external eye speculum of claim 1 wherein the length of the first arm and the second arm are generally curvilinear in shape.

7. An external eye speculum comprising:
a generally U-shaped member having a first arm and a second arm, the first and second arms joined at a point; the first arm and second arm each being of a predetermined length and further being generally curvilinear in shape with concave portions facing one another; an adhesive applied to at least a portion of the length of at least one of the first arm and the second arm; and, a first raised member extending generally upward direction from the first arm and a second raised member extending generally upward direction from the second arm.

8. The external eye speculum of claim 7 wherein the adhesive is tape.

9. The external eye speculum of claim 7 further comprising a handle extending in a generally outwardly direction from the point.

10. A method of use for an external eye speculum comprising the steps of:
removing a protective covering an adhesive found on at least a portion of a length of a first arm and a second arm, the first and second arms joined at a point, the first arm and second arm each being of a predetermined length and further having a first raised member extending generally upward direction from the first arm and a second raised member extending generally upward direction from the second arm; pinching the first and second raised members towards one another thereby causing the first arm and the second arm to move to a closed position thereby placing the external eye speculum over the target eye; releasing the pressure on the first raised member and second raised member thereby deploying the inventive device.

11. The method of claim 10 including the further step of reversibly adhering the adhesive to an eye lid and lash.
